(19) United States
(12) Patent Application Publication
(10) Pub. No.: US 2022/0073079 A1
(43) Pub. Date: Mar. 10, 2022

(54) ALCOLOCK DEVICE AND SYSTEM

(71) Applicant: GAZELOCK AB, Umeå (SE)

(72) Inventors: Anders NILSSON, Umeå (SE); Haibo LI, Täby (SE)

(73) Assignee: GAZELOCK AB, Umeå (SE)

(21) Appl. No.: 17/312,992

(22) PCT Filed: Dec. 12, 2019
(86) PCT No.: PCT/SE2019/051270

§ 371 Date: Jun. 11, 2021

(30) Foreign Application Priority Data
Dec. 12, 2018 (SE) 1830358-6

(51) Int. Cl. B60W 40/08; G06K 9/00; F02D 41/02

(52) U.S. Cl. CPC B60W 40/08; G06K 9/00845; G06K 9/00604; G06K 9/00288; B60W 2540/24; G06K 2009/00322; B60W 2540/225; B60W 2040/0836; F02D 41/021

(57) ABSTRACT

The present disclosure relates to an ignition interlock device and system for accurately detecting a drunk driver by running an interactive visual test presented for the driver to visualize on a screen of the alcolock device, which further comprises at least an eye gaze tracking module for recording eye movements and measuring gaze data, and a motor skill computing module for computing motion parameters from the sensor data measured during the interactive visual test. The alcolock device may further comprise a drunk detection module for measuring drunkenness of the driver by mapping gaze parameters and motion parameters to measure the mismatch between motor skills and cognitive processing performance, and a decision module for allowing the driver to drive the vehicle, or not, based on the measured drunkenness.

### TECHNICAL FIELD

The present technology relates to ignition interlock devices and systems, such as alcolock devices and systems.

### BACKGROUND

From the statistics of Finland, drunk driving is involved in 25% of fatal traffic accidents. Just in 2011, 74 persons died and 735 were injured in traffic accidents that involved drunk driving in Finland. It has been estimated that the cost of a traffic fatality is 1.9 million Euro. A permanent injury costs 1.0 million Euro and a temporary injury on average 241 000 Euro.

Furthermore, the statistics shows also that the profile of a drunk driver has not changed for a long period. About one third of drunk drivers are recidivists and the rate has remained at the same level for 30 years. The risk of being caught has not increased for 30 years. A drunk driver can still drive drunken about 220 occasions before being caught. The findings justify an obligatory use of Alcolocks as one preventive measure to counteract recidivism. Studies from Finland, Sweden, Canada and USA have shown good results of the impact of Alcolocks on recidivism. One of the serious problems with the existing Alcolocks is that they measure blood alcohol concentration through some kinds of breathing systems. Technically, such systems can be cheated easily in practice, for instance, one headache with Alcolock in a real application is to know if a driver cheats it by using a mask to filter his/her breathing airs.

One of the most common ways to determine if someone is under influence of some kind of drug, legal or illegal, is by looking them in the eyes. Dilated pupils, eye redness, nystagmus, problems with fixating gaze, all of these could be indicators for a person under the influence. The different impacts on the eyes are how we can take advantage of these visual cues to spot a drunk driver before they start driving. An efficient and effective way to do so is to use eye-tracking to measure if a driver is drunk.

More specifically, Alcohol ingestion will cause varying degrees of physiological losses that result in changes in the cognitive and behavioural functions as well as visual perception. The effects can be felt and measured even when alcohol is consumed in light to moderate levels. Intoxication due to occasional alcohol ingestion will affect the central nervous system (CNS). These effects of alcohol on the CNS result in alterations in the visual system that are related, for instance, to colour perception, contrast sensitivity, as well as on eye movements.

Eye movement is a good indicator of cognitive functions. One of the main functions of eye movements is to align information of potential interest and the fovea, thus selecting information from relevant parts of the visual environment. Therefore, eye movements are closely related to visual attention. Typical eye movements whilst scanning an image can be classifies as saccades and fixations. Saccades are ballistic movements of the eye itself from one point of the visual scene to another, whereas fixations refer to the time between the saccades in which the eye presents minimal movements.

There are some studies on the effect of alcohol intake on eye movement and visual perception and recognition. In it was concluded that alcohol dose affect human picture perception and decrease the performance of visual exploration. Another study showed that it is possible to see deviations in the gaze patterns of drunk people, even at a very low level of blood alcohol concentration. Measurement of eye movements has been suggested for detecting a drunk driver. However, it is not an effective and efficient way for drunk detection if patterns of eye movements and/or eye gaze are measured in some kind of passive way.

The study in showed that the time of the survey and the gender of the driver were high risk factors for drunk driving.

The following references provide information relating to alcolock devices and systems
1. Blincoe, L. J., Miller, T. R., Zaloshnja, E., & Lawrence, B. A. (2015, May). The economic and societal impact of motor vehicle crashes, 2010. (Revised)(Report No. DOT HS 812 013). Washington, D.C.: National Highway Traffic Safety Administration.
2. National Highway Traffic Safety Administration. Retrieved May 11 2018 from http://www.nhtsa.gov/risky-driving/drunk-driving.
3. Bergman, G, Larsson, A, Martinsson, A, Norén, F. (2018) The future of DUI detection technology,—A research study on prevention and methods for detecting drivers under the influence (DUI), KTH Media Lab Course Report.
4. Portman, M., Penttilä, A., Haukka, J., Rajalin, S., Eriksson, C., Gunnar, T., . . . Kuoppasalmi, K. (2013). Profile of a drunk driver and risk factors for drunk driving. Findings in roadside testing in the province of Uusimaa in Finland 1990-2008. Forensic Science International, 231(1-3), 20-27. doi:10.1016/j.forsciint.2013.04.010
5. Møller, M., Haustein, S., & Prato, C. G. (2015). Profiling drunk driving recidivists in Denmark. Accident Analysis & Prevention, 83, 125-131. doi: 10.1016 /j.aap. 2015
6. Dai, J., Teng, J., Bai, X., Shen, Z., & Xuan, D. (2010). Mobile phone based drunk driving detection. Proceedings of the 4th International ICST Conference on Pervasive Computing Technologies for Healthcare. doi: 10.4108/icst.pervasivehealth2010.8901
7. Driver Alcohol Detection System for Safety. https://www.dadss.org
8. Moser, A., Heide, W., & Köompf, D. (1998). The effect of oral ethanol consumption on eye movements in healthy volunteers. Journal of Neurology, 245(8), 542-550. doi:10.1007/s004150050240
9. Silva, J. B., Cristino, E. D., Almeida, N. L., Medeiros, P. C., & Santos, N. A. (2017). Effects of acute alcohol ingestion on eye movements and cognition: A double-blind, placebo-controlled study. Plos One, 12(10). doi:10.1371/journal.pone.0186061
10. Thien, N. H., & Muntsinger, T. Horizontal Gaze Nystagmus Detection in Automotive Vehicles.
11. GHO|By category|Legal BAC limits—Data by country. (n.d.). Retrieved May 10, 2018, from http://apps.who.int/gho/data/view.main.54600

### SUMMARY

According to a first aspect, it is in this disclosure provided an alcolock device for detecting a drunk driver by running an interactive visual test presented for the driver to visualize on a screen of the alcolock device, which further comprises a camera arrangement and physical sensors. The alcolock device further comprises at least an eye gaze tracking module for recording eye movements and measuring gaze data from which gaze parameters are extracted to characterize cognitive processing performance during the interactive visual test and a motor skill computing module for computing motion parameters from the sensor data measured during the interactive visual test. The alcolock device may further comprise a drunk detection module for measuring drunkenness of the driver by mapping gaze parameters and motion parameters to measure the mismatch between motor skills and cognitive processing performance and a decision module for allowing the driver to drive the vehicle, or not, based on the measured drunkenness.

According to further one aspect, it is in this disclosure provided an alcolock system for detecting a drunk driver intending to drive a vehicle, the alcolock system being configured to control the engine of the vehicle, wherein the alcolock system further comprises an alcolock device according to the first aspect.

### BRIEF DESCRIPTION OF THE DRAWINGS

The foregoing, and other, objects, features and advantages of the present invention will be more readily understood upon reading the following detailed description in conjunction with the drawings in which:

FIG. 1 is block diagram illustrating alcolock system according to the invention;

FIG. 2 is a scenario of the invention;

FIG. 3 is a block diagram of an alcolock device according to the invention;

FIG. 4 is a block diagram illustrating the determining of a large-scale dataset to be used by an eye gaze tracker of the alcolock device according to the invention;

FIG. 5 is a block diagram illustrating different movement directions according to the invention.

### DETAILED DESCRIPTION

The present technology relates to ignition interlock devices, or alcolock devices, and to ignition interlock systems or alcolock systems for vehicles. Alcolock is a registered trademark, but has been a common name for ignition interlock devices and systems, which is considered as the generic terms. Thus, an alcolock system used in this disclosure is herein similar to an ignition interlock system and an alcolock device is herein used in this disclosure similar to a to ignition interlock device. The alcolock device is a device for testing if a user is under influence of alcohol and/or drugs and if so, the alcolock device is configured to prohibit the use of a vehicle wherein the ignition interlock systems is installed and with which system the device is communicating. The device is a part of an alcolock, or ignition interlock, system. The alcolock device, i.e. ignition interlock device, is a user interface handled by a user who has the intention to drive a vehicle to which the alcolock device, i.e. ignition interlock device, and its alcolock system, i.e. ignition interlock system, belongs.

The alcolock system is configured to prohibit the use of a vehicle by a driver that is temporary not suitable for driving the vehicle. In this disclosure, the term “drunk driver” is used for a user or driver that is not suitable for driving a vehicle due to the fact that the driver or user has drunk alcohol or used any kind of narcotic drug or medicine that influences and decreases the driver's ability to drive safely in the traffic. The term driver should herein be interpreted as a user or person who wishes or intends to use the vehicle in question.

The present disclosure presents an alcolock device and an alcolock system, wherein the alcolock device is used for detecting a drunk driver by running an interactive visual test presented for the driver to visualize on a screen of the alcolock device, which further comprises a camera arrangement, physical sensors, e.g. for recording hand gestures, processor, memory and a computer operational system. The alcolock device further comprises at least an eye gaze tracking module for recording eye movements and measuring gaze data from which gaze parameters are extracted to characterize cognitive processing performance, a motor skill computing module for computing motion parameters from the sensor data measured under the visual interaction, and a drunk detection module for mapping gaze parameters and motion parameters to a measurement of drunkenness as a measure of the mismatch between motor skills and cognitive processing. The alcolock device and an alcolock system is hereafter described in more detail with reference to the attached drawings.

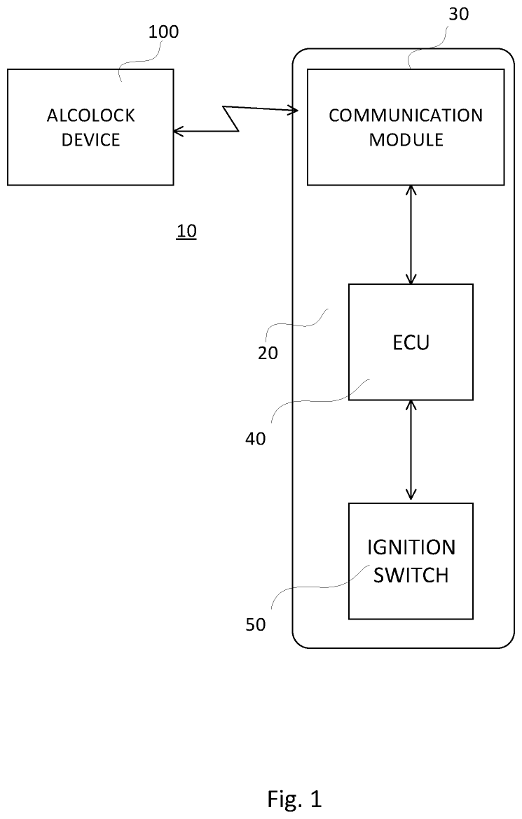

FIG. 1 is illustrating an alcolock system 10 for a vehicle. The alcolock system 10, i.e. ignition interlock system, comprises an alcolock device 100 and a vehicle control system 20. The vehicle control system 20 is installed in a vehicle. The alcolock device 100 is a unit that is handheld by the user or intended driver. The alcolock device 100 and the vehicle control system 20 are configured to communicate by signalling, either by wireless communication or via cable connection. The signalling may be digital or analogue.

Modern vehicles comprise a vehicle control system 20 that is controlling different functions of the vehicle, e.g. the ignition circuitry of the vehicle, the electric motor driving circuitry of the fuel pump, power circuitry from electric batteries feeding the electric engine with electric power, etc.

According to the in FIG. 1 illustrated example of a vehicle control system 20, said system may comprise a communication module 30, an Engine Control Unit, ECU, 40 and an ignition switch 50. According to another embodiment, the ignition switch 50 is replaced by a switch 50 operating on a vehicle electric system.

In a fuel driven vehicle, the Engine Control Unit 30 may be connected for controlling an electric system, e.g. the ignition circuitry of the vehicle or the electric motor driving circuitry of the fuel pump. In an electrically driven vehicle, the ECU 40 may be connected to an electric system, e.g. the power circuitry from electric batteries feeding the electric engine with electric power, or connected to an electrically or electronically controlled switch located in the electric power feed system between the electric battery and the electric motor of the vehicle.

The alcolock device is configured to directly or indirectly control the operation of engine of the vehicle. This may be achieved by controlling the control system 20 so as to lock and/or unlock the operation of the vehicle's engine by controlling the whole or a part of an electric system of the vehicle.

The ECU 40 of the vehicle control system 20 may be configured to operate in two different states—driving locked or driving unlocked. In the driving locked state, the ECU locks the vehicle by disabling the operation of the whole or a part of the electric system of the vehicle. In the driving locked state, the vehicle cannot be started and driven by an intended driver, i.e. a person that has the intention to drive the vehicle. In the driving unlocked state, the vehicle can be started and driven by a driver.

Preferably, in a first example, the ECU 40 is in its driving locked state when the vehicle is not used which condition forces the driver to use the alcolock device 100 to try to switch the state of the ECU. According a second example, the ECU 40 is in its unlocked state when the vehicle is not used, the driver may have the option to use the alcolock device to test if he or she is capable of driving the vehicle. If the driver is tested by the alcolock device to be not suitable for driving the vehicle, the alcolock device switches the ECU from driving unlocked to driving locked state.

The alcolock device 100 is configured to send a drive locking signal or drive releasing signal to the vehicle control system 20, of which the system communication module 30 receives the signal and forwards it to the Engine Control Unit, ECU, 40 that controls e.g. an ignition switch 50.

If a drive locking signal is received by the ECU 40, the ECU switches to or remains in its driving locked state, and the ECU locks the vehicle by disabling the operation of the whole or a part of the electric system of the vehicle, e.g. by means of the ignition switch 50.

The driving locked state will remain until the alcolock device 100 signals to the ECU to switch state from driving locked to driving unlocked state.

When a drive releasing signal is received by the ECU 40, the ECU switches to or remains in its driving unlocked state, and the engine of the vehicle is possible to start by means of the ignition switch 50 and the driver will be able to drive the vehicle.

This disclosure presents a new approach to build alcolocks, i.e. ignition interlock systems, based on the fact that drinking alcohol will impair both motor skills and cognitive functioning of a person. The fact can be used to detect if a person is drunk through measuring his/her motor skills and cognitive functions.

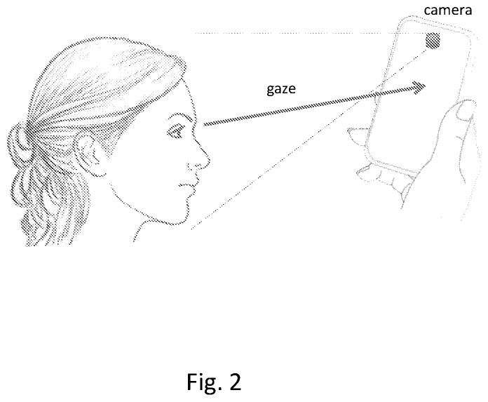

The application scenario is shown here in FIG. 2. To unlock a vehicle, e.g. a car, lorry, etc, the driver needs to orient the device by the hand and/or use the touch screen to finish a visual test running in the screen of the device. The driver is instructed to hold the device in her/his hand and to run an interactive visual test by moving the device in accordance to the interactive visual test. Eye movements are recorded through a camera arrangement and an eye gaze tracker of the alcolock device.

Since the intake of alcohol will cause transient motor and cognitive changes, when performing an instruction, e.g. a visual searching task, the user needs a large number and duration of fixations, high latency for the initial fixation and a high number of saccades, as well as a high total time. As an indicator of cognitive processing, eye movement can be used to measure the effects of alcohol or drug intake.

In the disclosure is presented, a robust way to accurately detect drunk person intending to drive a vehicle or drivers. To unlock a vehicle, the driver has to run an interactive visual test which has a high demand of cognitive function performance and motor skills. Unlike other approaches, it is not a simple measurement of eye movement rather but measurement of deviation in the gaze patterns in a closed hand-eye coordination process. Gaze patterns may be described and defined by means of gaze parameters. More specifically, it is not just the performance of the cognitive functioning that are measured through eye movement but the mismatch between the cognitive functions performance and motor skills that is measured.

Our principle is based on the fact that drinking alcohol can impair both motor skills and cognitive functioning, but motor skills can be re-gained at a faster rate than the performance of the cognitive functioning. Cognitive function is also denoted as cognitive processing or cognitive skill. This could create the illusion of complete sobriety and prompt the undertaking of activities requiring cognitive processing that are still greatly impaired. This will result in fatal mistakes, for example, problem to make correct responses very fast. An example of such a mistake is to press the accelerator pedal rather than the brake pedal in an emergency situation. Therefore, the most effective way is to measure the mismatch between motor skills and cognitive functions, i.e. the mismatch between motor skill and cognitive processing performance or cognitive skill of a person. The mismatch is a more sensitive effect than use of cognitive functions alone for detecting drunk drivers. To compute the mismatch, a way of measuring motor skills in an interactive visual test process is proposed herein. A person that wants to drive has to hold an alcolock device or a mobile phone to run a designed visual test. His/her motor skills can be measured through physical motion sensors embedded in the alcolock device, or through once already existing in modern mobile phones. This is different from some existing approaches of using mobiles to combat drunk driving [6]. In these known approaches mobile phones, the accelerometer and orientation sensors are used to detect patterns associated with driving under the influence of alcohol and drugs. The sensor generated data are used to compute driving behaviours but not for measuring personal motor skills in a visual test.

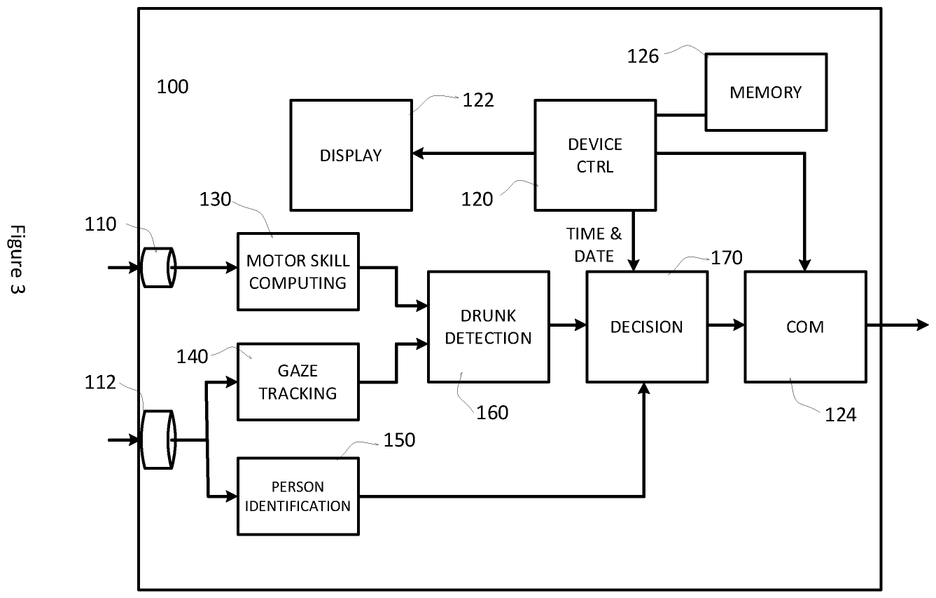

FIG. 3 is a block diagram of an alcolock device according the new approach and aspect described in this disclosure.

The alcolock device 100 has a screen or display 122 on which a designed visual test is presented for the driver to visualize. The screen and display may be of the touch-screen type. Besides the screen 122 the device comprises a camera arrangement 112 comprising one or more video cameras, one or more physical sensors 110, a digital processor 120, memory 126 for storing data and computer operational system, and communication module 124. The digital processor 120 is configured to control the operation of the screen 122, camera arrangement 112, physical sensors 110, and communication module 124.

The camera arrangement 112 may be a video camera or an array of video cameras that is capable of capturing and storing face images of the driver. The output will be a face video or multiple face videos.

The alcolock device 100 may further comprise at least a number of the following technical modules: a person identification module 150, an eye gaze tracking module 140, a motor skill computing module 130, a drunk detection module 160, and a decision module 170. Some or all of the modules may be implemented by means of computer software executed by one or more digital processors or processing circuitry or deep learning networks.

The eye gaze tracking module 140 is used for recording eye movements and measuring gaze data from which gaze parameters are extracted by computing means to characterize cognitive processing performance during the visual interaction test of the driver or user.

The motor skill computing module 130 is used for computing motion parameters from the sensor data measured during the visual interaction test of the driver or user.

The drunk detection module is configured for measuring, i.e. computing, drunkenness as a probability (s) of the driver by mapping gaze parameters and motion parameters to measure the mismatch between motor skills and cognitive processing performance.

The person identification module 150 is used for capturing by means of the camera arrangement a face image of the driver for identifying the personal identity. The person identification module 150 may further be configured to estimate identity, gender, age information from the captured face image.

The person identification module 150 may further be configured to retrieve sensitive personal socioeconomic status information from remote databases or databases in the cloud where personal socioeconomic status information has been stored. The person identification module 150 may be configured to by means of the communication module 124 retrieve personal socioeconomic status information from said database or databases.

Examples of such information associated with the driver may be if the driver is divorced, widowed, unemployed, a recidivism or a suspected/known terrorist or if the driver has a legal driving license. The person identification module 150 is configured to identify the user or driver, and if the user or driver is a suspected/known terrorist or if the driver does not have a legal driving license, the person identification module 150 indicates to the decision module 170 that the alcolock system should be locked prohibiting that the vehicle to be started and used by the identified person. Thus, besides stopping drunk driving, it is an option to prevent the misuse of the car by someone else for a terror attack even though the driver is found to be sober.

Furthermore, a desired option for a working alcolock system is to make sure that the person who has successfully unlocked the alcolock system should be the same one who is driving the vehicle. This may be achieved by putting the alcolock device in front of the driving seat in a fixed holder arrangement before starting the vehicle as to identify who is sitting in the driving seat when the vehicle is started and beginning to move. If the person in the driving seat is identified by the person identification module 150 to be the same person who unlocked the alcolock system, the alcolock system will remain unlocked. If the person in the driving seat is identified by the person identification module 150 to not be the same person who unlocked the alcolock system, the alcolock system will quickly change from unlocked to locked and stop the engine from running prohibiting the further movement of the vehicle. As the vehicle has just begun to move, the speed is very low, and a stop is safe.

Thus, the camera arrangement capture the face image of the driver for identifying the personal identity by the person identification module. The obtained identity will be used to retrieve the sensitive personal socioeconomic status information like if the driver is divorced or widowed, or unemployed, or a recidivism. Besides the identity, gender, age information can be estimated from the face image. All kind of personal information is used to aid the final decision of if the driver is drunk. The identified identity is used to check if the driver is driving the car later on (make sure it is the same person).

The alcolock device and system may therefore be configured to integrate personal socioeconomic status information, time and date into a final decision of drunkenness and/or allowability to drive the vehicle.

Besides the technical parts, another component in the system is the design of interactive visual test. The used test should be a relatively simple task for the tested person, i.e. the driver, wherein the task should be more resistant to psychosocial factors and individual differences. The problem resolution in the test requires cognitive processing of the test person, in which processes operate such as flexibility, inhibitory control, attention, planning, visual attention and decision making, and in an integrated hand eye coordination manner, allowing the individuals to guide operation behaviour to the goals and solve problems.

The output of the person identification module 150 includes personal identity and other attributes like gender, age. The identity will further retrieve the sensitive personal socioeconomic status information and send the information to the decision module 160.

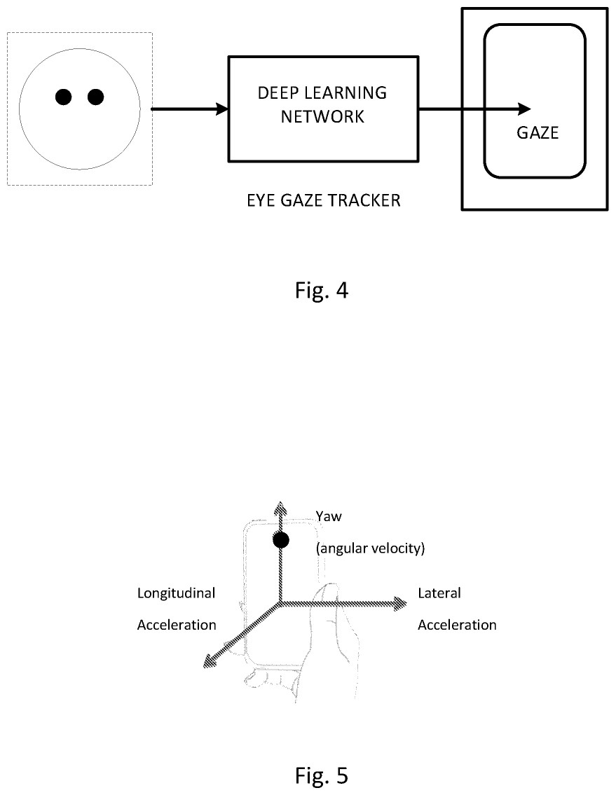

FIG. 4 is a block diagram illustrating the determining of a large-scale dataset to be used by an eye gaze tracker of the alcolock device.

The eye gaze tracking module 140 may be implemented as a deep learning network for constituting a high-accuracy, calibration-free eye gaze tracker by training with a large-scale dataset. The approach is to use the visual information from one or more face images to robustly predict eye gaze directly as shown in FIG. 4. To achieve such an end-to-end mapping from a face image to gaze, a deep neural networks is used to make an effective use of a large-scale dataset. This is a direct way of mapping a full face image by an end-to-end deep learning network to gaze coordinates. To achieve such a direct mapping, one has to first select a deep learning network, for example, using the ResNet model with batch normalization, and then to train the network. To train the network, one needs to collect a large-scale eye tracking dataset which should contain face images from more than ten thousand unique test subjects captured by a mobile phone, like iPhone or android phone, or even an iPad. The face images are labelled with an (x, y) coordinate corresponding to the point on the screen that the user looked at when the photo or video is taken. The deep learning network model is then trained by minimizing a regression loss using the collected dataset. After the training, the network can be used to achieve a direct mapping from a full face image to the (x, y) coordinate of the user's gaze. FIG. 4 indicates the structure of the full eye gaze tracker deep learning network.

For achieving high-accuracy, calibration-free eye gaze tracking, the learning of a robust eye gaze tracking model from significant variability in the data is performed. The large-scale database plays an important role in the modelling, wherein the training data is collected in a large variability in pose, appearance, and illumination. Eye gaze tracking end-to-end is learnt based on machine learning algorithms without the need to include any manually engineered features, such as head pose.

During the running of the visual test, the cognitive processing performance of the test person (driver) is measured by using the following 6 gaze parameters extracted from the gaze data to characterize cognitive processing performance or cognitive function:

1) Latency of first fixation (FF);

2) Number of fixation (NF)

3) Duration of fixation (DF)

4) Number of saccades (NS)

5) Duration of saccades (DS)

6) Task execution time (TE).

The gaze parameters are used for measuring both speed and accuracy of cognitive performance. For example, a high number and time of fixations negatively correlate with the efficiency of a visual test search. In addition, the less time to first fixation, indicating impaired reaction time and attention orientation. The drunk driver under the influence of alcohol usually shows less efficiency in cognitive processing during the resolution of the problem proposed in the visual test. The saccadic movements are closely linked to the visual attention. Alcohol can affect the attention control and reduce the accuracy of location visual targets. The individuals under the effect of alcohol need to perform a greater sweep of sequential elements to set the goal oriented behaviours.

The 6 gaze parameters are sent as input to the drunk detection module 160 for further processing together with 3 motion parameters received from the motor skills module during the visual test period.

To measure motor skills of the driver, the following physical sensors 110 may be used:
Proximity sensor
Accelerometer
Gyroscope
Compass

Said sensors 110 may be embedded into the alcolock device 100 and they are used for recording hand gestures. A proximity sensor is a sensor able to detect the presence of nearby objects without any physical contact. A signal of the absence or presence of objects will be outputted. The signal is used for activating the alcolock device.

An accelerometer is a device that measures proper acceleration of the handheld device. The signal of magnitude and direction of the proper acceleration will be outputted. A gyroscope is a device used for measuring or maintaining orientation and angular velocity. The orientation and angular velocity of the device is sent to the motor skill computing module. A compass is a device for detecting and measuring magnetic fields, which outputs the direction relative to the geographic cardinal directions.

For determining the motor skills of a driver, the motion skills under the visual interaction can be indirectly measured through the dynamics of the device. The motion skill is the motion behaviour of the user's hand holding the device. To play the designed visual test, e.g. a game visualized on the display of the alcolock device, the user or intended driver has to move the alcolock device in the air to finish the game. The motion behaviours are recorded as dynamics of the alcolock device and used for motion analysis.

The dynamics of the alcolock device will be specified by three motion parameters: the longitudinal and lateral accelerations of the device as well as its yaw or angular velocity in the vertical axis, as illustrated in FIG. 5. These three parameters are highly related to the motor skill of a driver. The three accelerations and angular velocity parameters in the referential of the device are computed from sensor data to derive the gesture behaviours during the run of the visual test wherein the driver interacts with the alcolock device.

If a mobile phone is used instead of a device, the algorithms can directly run over the mobile phone where the above mentioned physical sensors is embedded to derive the motion parameters of the gesture behaviours.

The drunk detection module 160 may be implemented as a deep learning network to achieve a direct mapping from six gaze parameters and three device motion parameters to a measurement of drunkenness as a measure of the mismatch between motor skills and cognitive processing.

When a driver drives a car, he/she uses both motor skills and cognitive processing skills. The motor and cognitive skills are synchronized in a hand-eye coordination way. The hand-eye coordination can be characterized in a high-dimensional space spanned by features extracted from both motor and cognitive skills. More specifically, a drunk detection module of the alcolock device extracts three motion parameters and six gaze parameters and embed them in a nine dimensional feature space to form a subspace of “non-drunk”. As long as a driver is not drunk, the extracted 9 motion and gaze parameters will stay in the subspace of “non-drunk”.

When a driver is drunk, drinking alcohol can impair both motor skills and cognitive functioning, but as one sobers up he/she regains motor skills at a faster rate than his cognitive function, which could give him a false sense of security. Technically, the motor system and cognition system of the driver are no longer synchronized and the hand-eye coordination system is broken. The nine motion and gaze parameters will go out the subspace of “non-drunk” to form a new subspace of “drunk” in the nine dimensional space. The drunk detection module can geometrically and algorithmically distinguish these two subspaces through a deep-learning process. In this way one can detect if a driver is drunk based on the 9 motion and gaze parameters.

The function of the decision module 170 is to make the final decision if the driver is drunk based on the 9 motion and gaze parameters.

The module 170 may be configured to employ a Bayesian framework to evaluate an evidence s received from the drunk detection module 160. One of the important task of this module is to compute the LR (likelihood ratio). The LR is the ratio of the distribution of this random variable s under two hypotheses evaluated at the realized value of evidence:

LR(s)=P(s|H_{d})/P(s|Hₙ)

where s is the evidence which is, the measurement of drunkenness. P is a Probability Density Function (pdf). H_{d} and Hₙ are two mutually exclusive and exhaustive hypotheses defined as follows:

H_{d}: The driver is drunk.

Hₙ: The driver is not drunk.

The LR calculates a conditional probability of observing a particular value of evidence s with respect to H_{d} and Hₙ. It is a concept, which provides for evaluation and comparison of the two hypotheses concerning the likely source of the data obtained from the drunk detection module 160. Once the LR of the drunkenness is computed, it can be interpreted as the multiplicative factor which update prior (before observing evidence from other modules) belief to posterior (after obtaining evidence from the drunk detection module) belief using the Bayesian framework:

P(H_{d}|s)/P(Hₙ|s)=P(s|H_{d})/P(s|Hₙ) P(H_{d})/P(Hₙ)

In this framework, the decision module is responsible for quantification of prior beliefs about H_{d} and Hₙ for the driver (the personal information and time and date are used to compute the prior beliefs) while the drunk detection module 160 is responsible for quantification of evidence in the form of the LR given the evidence. It is clear from the definition of the LR that the distribution of evidence should be considered given the two hypotheses H_{d} and Hₙ. The job of drunk detection module 160 is to express the evidence in relation to distribution of evidence given two competing hypotheses while the job of the decision module 170 is to assess the posterior probabilities of the two competing hypotheses given the evidence.

Besides safety personal identity is also very helpful in making alcolocks more robust. The risk on a Saturday morning was about eight times higher than during Tuesday afternoon. The risk for a female to drive drunk was less than one fifth of that for men. Divorced and widowed people had a clearly higher risk than married drivers. In the age group ‘30-54 years’ the risk for drunk driving was higher compared to the age group below 20 years. Unemployed drunk drivers had also higher blood alcohol concentration. Therefore, the context and personal socioeconomic status will be very useful in aiding the detection of drunk driver.

The personal information and time and date will be integrated into the final decision of drunk drivers to make sure, for example, that a higher threshold is set to woman than man. The time of the day and the date may be requested and received from the device controller 120.

The decision module 170 is to employ the Bayesian framework to integrate evidence and propagate the integrated belief. Before the evidence of the drunkenness is computed and integrated, the prior belief has been computed and updated from the additional information, like, personal attributes, time and date information, that is, the additional information is used for quantification of prior beliefs about Ha and Hn for the driver. The prior belief is then updated by integrating the evidence to posterior belief (after obtaining evidence from the drunk detection module 160) belief using the Bayesian framework.

With reference to FIGS. 1 and 3, the operation of the alcolock system 10 for detecting a drunk driver intending to drive a vehicle is hereafter described.

The alcolock system 10 is configured to control the starting of an engine of the vehicle by means of an alcolock device 100.

A driver intending to start and drive the vehicle has to pick up the alcolock device 100 (or mobile phone) and gaze on the screen 122. The alcolock device then automatically starts (due to a signal from the proximity signal) and one or more video cameras of a camera arrangement 112 start capture or record images of the driver's face. The driver is identified by means of the person identification module 150 using a face image.

In the next step, an interactive visual test is run on the screen 122 of the alcolock device 100 during a test period. The driver has the alcolock device in one hand. The test is designed such as the problem resolution in the test requires cognitive processing of the driver, in which processes operate such as flexibility, inhibitory control, attention, planning, visual attention and decision making, and in an integrated hand eye coordination manner, allowing the diver to guide operation behaviour to at least one goal and solve different problems.

During the interactive visual test period, the motor skill computing module 130 receives from the accelerometer, gyroscope and compass sensors 110 signals for determining values of three motion parameters over time as a measure of the motor skill, and the eye gaze tracking module 140 receives from the camera arrangement one or more face images of the driver. The eye gaze tracking module 140 is configured to analyse the received images for determining the six gaze parameters. The three motion parameters and the six gaze parameters constitute input values to the drunk detection module 160. It is not just cognitive functions that are measured through eye movement by the module 140, it is the mismatch between the driver's cognitive function and motor skills that is measured.

The drunk detection module 160 may be implemented as a deep learning network to achieve a mapping of the six gaze parameters and three motion parameters as a measurement of drunkenness s, also denoted as the evidence s. The decision module is configured for allowing the driver to drive the vehicle, or not, based on the measured drunkenness.

The evidence is input to the decision module 170. The decision module assess the posterior probabilities of two competing hypothesis given the measurement of drunkenness. Besides the evidence, the decision module 170 may also integrate additional information in the decision process, e.g. time and date when the test is performed as well as at least one of the following information: identity, gender, age and personal socioeconomic status of the driver to be able to improve the decision whether the driver should drive or not.

If the decision is that the driver is not suitable for driving the vehicle, the decision module 170 is configured to generate a first signal, a drive locking signal. If the decision is that the driver is suitable for driving the vehicle, the decision module 170 is configured to generate a second signal, a drive releasing signal. The drive locking signal and drive releasing signal are fed as input to a communication module 124. The communication module 124 sends the drive locking signal or drive releasing signal to the vehicle control system 20, of which the system communication module 30 receives the signal and forwards it to the Engine Control Unit, ECU, 40 that controls e.g. an ignition switch 50.

If a drive locking signal is received by the ECU 40, the ECU switches to or remains in its driving locked state, and the ECU locks the vehicle by disabling the operation of the whole or a part of the electric system of the vehicle, e.g. by means of the ignition switch 50.

If a drive releasing signal is received by the ECU 40, the ECU switches to or remains in its driving unlocked state, and the ECU the engine of the vehicle is possible to start by means of the ignition switch 50 and the driver will be able to drive the vehicle.

A number of embodiments of the alcolock device and alcolock system have been described. It will be understood that various modifications may be made without departing from the scope of the following claims.

What is claimed is:

1.-11. (canceled)

12. An alcolock device for detecting a drunk driver; the alcolock device comprising:
a camera arrangement, physical sensors, processor, memory and a computer operational system, the alcolock device further comprising at least:
an eye gaze tracking module for recording eye movements and measuring gaze data from which gaze parameters are extracted to characterize cognitive processing performance during the interactive visual test;
a motor skill computing module for computing motion parameters from the sensor data measured during the interactive visual test; and
a drunk detection module for measuring drunkenness of the driver by mapping gaze parameters and motion parameters to measure the mismatch between motor skills and cognitive processing performance; and
a decision module for allowing the driver to drive the vehicle, or not, based on the measured drunkenness.

13. The alcolock device according to claim 12, wherein the interactive visual test is designed such as the problem resolution in the test requires cognitive processing, in which processes operate such as flexibility, inhibitory control, attention, planning, visual attention and decision making, and in an integrated hand eye coordination manner, allowing the individuals to guide operation behaviour to the goals and solve problems.

14. The alcolock device according to claim 12, wherein the gaze parameters are at least one of:
Latency of first fixation (FF);
Number of fixation (NF);
Duration of fixation (DF);
Number of saccades (NS);
Duration of saccades (DS); and
Task execution time (TE).

15. The alcolock device according to claim 12, wherein the motion parameters are the three device motion parameters in lateral and longitudinal accelerations and angular velocity parameters

16. The alcolock device according to claim 12, wherein the eye gaze tracking module for measuring a cognitive processing performance of the driver is a deep learning network providing a high-accuracy, calibration-free eye gaze tracker by training with a large-scale dataset.

17. The alcolock device according to claim 12, wherein drunk detection module is a deep neural network.

18. The alcolock device according to claim 12, further comprising:
a person identification module for capturing a face image of the driver for identifying the personal identity.

19. The alcolock device according to claim 18, wherein the person identification module is configured to estimate at least one of identity, gender, and age information from the face image.

20. The alcolock device according to claim 19, wherein the person identification module retrieves the sensitive personal socioeconomic status information like if the driver is divorced or widowed, or unemployed, or a recidivism.

21. The alcolock device according to claim 20, wherein the alcolock device integrates personal socioeconomic status information, time and date into a final decision of drunkenness.

22. An alcolock system for detecting a drunk driver intending to drive a vehicle, the alcolock system being configured to control the engine of the vehicle, wherein the alcolock system further comprises an alcolock device according to claim 12.
